(19) United States
(12) Patent Application Publication
(10) Pub. No.: US 2023/0183198 A1
(43) Pub. Date: Jun. 15, 2023

(54) METHODS OF PRODUCING GLYCIDYL NITRATE AND RELATED SYSTEMS

(71) Applicant: Northrop Grumman Systems Corporation, Falls Church, VA (US)

(72) Inventors: Christina N. Christensen, Hooper, UT (US); Joseph J. Scavuzzo, Wadsworth, OH (US); Melissa L. Mileham, Providence, UT (US)

(21) Appl. No.: 18/063,841

(22) Filed: Dec. 9, 2022

Related U.S. Application Data
(60) Provisional application No. 63/288,451, filed on Dec. 10, 2021.

(51) Int. Cl. C07D 303/36; B01J 19/00

(52) U.S. Cl. CPC C07D 303/36; B01J 19/0093

(57) ABSTRACT

A method of producing glycidyl nitrate comprises reacting a glycerol solution and nitric acid in a microfluidic reactor to form a dinitroglycerol solution. The glycerol solution exhibits a viscosity of less than or equal to about 150 cP at about 20° C. The dinitroglycerol solution is reacted with a base in the microfluidic reactor to form glycidyl nitrate. Related systems and methods are also disclosed.

### CROSS-REFERENCE TO RELATED APPLICATION

This application claims the benefit under 35 U.S.C. § 119(e) of U.S. Provisional Patent Application Ser. No. 63/288,451, filed Dec. 10, 2021, the disclosure of which is hereby incorporated herein in its entirety by this reference.

### TECHNICAL FIELD

Embodiments of the disclosure relate generally to methods of producing an energetic material. More particularly, embodiments of the disclosure relate to methods of producing glycidyl nitrate (GLYN) by a microfluidic process.

### BACKGROUND

Glycidyl nitrate (GLYN) is an energetic precursor that is used to produce poly(glycidyl nitrate) (PGN), an energetic polymer. The PGN is used as a polymer in a binder system for explosives or propellants. Producing GLYN is a hazardous process that includes two exothermic chemical reactions and generates trinitroglycerol (NG) as a byproduct. Glycerol is nitrated to form a dinitroglycerol (DNG) compound, which is cyclized to form the GLYN. During the nitration reaction, process conditions are closely controlled to prevent runaway reactions. Extensive cooling is also utilized to control the reaction. The thermally unstable oxirane ring of GLYN also contributes to the hazards of the process.

Conventionally, GLYN has been produced by batch or continuous batch processes. The batch processes have a low capital cost and a reasonable ability to scale up. However, the hazards associated with the batch processes are high due to the large volumes and amounts of reagents, intermediates, and reaction products used in large scale production. The conventional processes also use large volumes of solvent, such as dichloromethane (DCM), that subsequently need to be disposed of. The continuous batch processes have a high capital cost and a reasonable ability to scale up, along with a high likelihood of hazards due to the large volumes and amounts of reagents, intermediates, and reaction products used for large scale production. Mitigating the hazards in the batch and continuous batch processes has made conventional processes of producing GLYN prohibitively expensive. The large scale production of GLYN is, therefore, not only dangerous but also expensive.

### BRIEF SUMMARY

A method of producing glycidyl nitrate according to embodiments of the disclosure comprises reacting a glycerol solution and nitric acid in a microfluidic reactor to form a dinitroglycerol solution. The glycerol solution exhibits a viscosity of less than or equal to about 150 cP at about 20° C. The dinitroglycerol solution is reacted with a base in the microfluidic reactor to form glycidyl nitrate.

A method of producing glycidyl nitrate according to other embodiments of the disclosure comprises reacting an aqueous glycerol solution and nitric acid in a microfluidic reactor at a temperature between about 15° C. and about 55° C. to form a dinitroglycerol solution. The dinitroglycerol solution is reacted with an aqueous potassium hydroxide solution in the microfluidic reactor at a temperature between about 20° C. and about 60° C. to form glycidyl nitrate.

A system for producing glycidyl nitrate is disclosed and comprises a microfluidic reactor comprising one or more inlets configured to introduce diluted glycerol, nitric acid, potassium hydroxide, and dichloromethane into channels thereof. The diluted glycerol exhibits a viscosity of less than or equal to about 150 cP at about 20° C. One or more liquid:liquid phase separators is coupled to the microfluidic reactor and is configured to remove nitric acid from a biphasic dinitroglycerol solution that comprises dinitroglycerol, nitric acid, dichloromethane, and water. One or more additional liquid:liquid phase separators is coupled to the microfluidic reactor and is configured to recover glycidyl nitrate from a biphasic glycidyl nitrate solution comprising glycidyl nitrate and dichloromethane.

### BRIEF DESCRIPTION OF THE DRAWINGS

FIG. 1 is a schematic illustration of a microfluidic reactor configured to produce GLYN in accordance with embodiments of the disclosure;

FIG. 2 is a schematic illustration of a microfluidic reactor configured to perform nitration of glycerol in accordance with embodiments of the disclosure;

FIG. 3 is a graphical representation of nitration results associated with glycerol nitration samples processed in the microfluidic reactor of FIG. 2;

FIG. 4 is a graphical representation of mononitroglycerol (MNG) and NG concentration in a product stream after nitration of glycerol in the microfluidic reactor of FIG. 2 plotted against reaction temperature at a constant nitric acid to glycerol ratio of 4:1;

FIG. 5 is a schematic illustration of a microfluidic reactor configured to produce GLYN in accordance with embodiments of the disclosure;

FIG. 6 is a schematic illustration of a microfluidic reactor configured to produce GLYN in accordance with embodiments of the disclosure;

FIG. 7 is a schematic illustration of a microfluidic reactor configured to perform nitration of glycerol and pre-separation of nitric acid from a nitration product stream in accordance with embodiments of the disclosure; and

FIG. 8 is a schematic illustration of a microfluidic reactor configured to produce GLYN in accordance with embodiments of the disclosure.

### DETAILED DESCRIPTION

Methods of producing glycidyl nitrate (GLYN) are disclosed. The GLYN is produced and separated by a continuous process using a microfluidic reactor (e.g., a flow reactor) having a continuous flow reaction channel with an inner diameter of less than or equal to about 1000 μm and a reaction volume of less than or equal to about 40 ml. Diluted glycerol is reacted with a nitrating agent to form a nitrated glycerol compound, which is subjected to an intramolecular condensation in the presence of a base to produce the GLYN. Unreacted nitrating agent is removed from a solution of the nitrated glycerol compound before conducting the intramolecular condensation reaction. The nitrated glycerol compound is separated from the solution by liquid:liquid separation techniques before conducting the intramolecular condensation reaction. The GLYN is recovered from a solution by liquid:liquid separation techniques. The nitration reaction, the intramolecular condensation reaction, and the separations are conducted inline using a system that includes the microfluidic reactor, enabling the GLYN to be produced continuously. The GLYN produced according to embodiment of the disclosure is in a substantially purified form compared to the GLYN produced by conventional GLYN processes. Since the reactions and the separations are conducted in a single system, the GLYN is produced continuously. The GLYN is produced by a safer and quicker process than conventional batch or continuous processes. Short reaction times may significantly reduce the amount of time needed to produce a desired amount of the GLYN. The conversion from glycerol to GLYN according to embodiments of the disclosure is comparable to conventional batch or continuous processes of producing GLYN.

As used herein, the singular forms “a,” “an,” and “the” are intended to include the plural forms as well, unless the context clearly indicates otherwise.

As used herein, the term “about” or “approximately” in reference to a numerical value for a particular parameter is inclusive of the numerical value and a degree of variance from the numerical value that one of ordinary skill in the art would understand is within acceptable tolerances for the particular parameter. For example, “about” or “approximately” in reference to a numerical value may include additional numerical values within a range of from 90.0 percent to 110.0 percent of the numerical value, such as within a range of from 95.0 percent to 105.0 percent of the numerical value, within a range of from 97.5 percent to 102.5 percent of the numerical value, within a range of from 99.0 percent to 101.0 percent of the numerical value, within a range of from 99.5 percent to 100.5 percent of the numerical value, or within a range of from 99.9 percent to 100.1 percent of the numerical value.

As used herein, the term “and/or” includes any and all combinations of one or more of the associated listed items.

As used herein, spatially relative terms, such as “beneath,” “below,” “lower,” “bottom,” “above,” “upper,” “top,” “front,” “rear,” “left,” “right,” and the like, may be used for ease of description to describe one element's or feature's relationship to another element(s) or feature(s) as illustrated in the figures. Unless otherwise specified, the spatially relative terms are intended to encompass different orientations of the materials in addition to the orientation depicted in the figures. For example, if materials in the figures are inverted, elements described as “below” or “beneath” or “under” or “on bottom of” other elements or features would then be oriented “above” or “on top of” the other elements or features. Thus, the term “below” can encompass both an orientation of above and below, depending on the context in which the term is used, which will be evident to one of ordinary skill in the art. The materials may be otherwise oriented (e.g., rotated 90 degrees, inverted, flipped, etc.) and the spatially relative descriptors used herein interpreted accordingly.

As used herein, the terms “comprising,” “including,” “containing,” “characterized by,” and grammatical equivalents thereof are inclusive or open-ended terms that do not exclude additional, unrecited elements or method steps, but also include the more restrictive terms “consisting of” and “consisting essentially of” and grammatical equivalents thereof.

As used herein, the term “configured” refers to a size, shape, material composition, and arrangement of one or more of at least one structure and at least one apparatus facilitating operation of one or more of the structure and the apparatus in a pre-determined way.

As used herein, the term “diluted glycerol” means and includes a solution of glycerol and a solvent, the solution having a lower concentration of glycerol relative to neat glycerol.

As used herein, the term “dinitroglycerol compound” or “DNG compound” means and includes 1,2-dinitroglycerol, 1,3-dinitroglycerol, or a combination thereof. The term “1,2-DNG” refers to 1,2-dinitroglycerol and the term “1,3-DNG” refers to 1,3-dinitroglycerol.

As used herein, the term “may” with respect to a material, structure, feature or method act indicates that such is contemplated for use in implementation of an embodiment of the disclosure and such term is used in preference to the more restrictive term “is” so as to avoid any implication that other, compatible materials, structures, features and methods usable in combination therewith should or must be excluded.

As used herein, the term “microfluidic reactor” means and includes a vessel (e.g., a reactor) configured to conduct chemical reactions under geometrically constrained conditions. The reactor includes a reaction channel having internal dimensions on the p.m scale, such as between about 1 μm and about 1000 μm, and a reaction volume of less than or equal to about 40 ml. For example, the reaction channel may have an inner diameter of less than or equal to about 1000 μm. The reaction volume of the microfluidic reactor may, however, be increased if an amount of GLYN to be produced is scaled up.

As used herein, the term “ratio” means and includes a relative magnitude of a flow rate of one reagent or solvent to a flow rate of another reagent or solvent, unless otherwise specified.

As used herein, the term “reaction solution” means and includes a combination of reagents (e.g., the glycerol and nitrating agent, the nitrated glycerol compound and base) in an optional solvent. The reagents (e.g., the glycerol and nitrating agent, the nitrated glycerol compound and base) may be substantially soluble in the optional solvent, may be substantially miscible with one another, or may be substantially immiscible with one another.

As used herein, the term “reaction volume” means and includes a volume of a reaction channel of the microfluidic reactor within which the reaction(s) is conducted.

As used herein, the term “residence time” means and includes a total time the reaction solution including the reagents is in the microfluidic reactor. The residence time is a function of the reaction volume of the microfluidic reactor and of a flow rate or ratio of flow rates that the reaction solution is flowed through the microfluidic reactor. The residence time corresponds to the amount of time for the reaction volume to move through the microfluidic reactor.

As used herein, the term “substantially,” in reference to a given parameter, property, or condition, means to a degree that one of ordinary skill in the art would understand that the given parameter, property, or condition is met with a small degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

The following description provides specific details, such as reagents, reagent amounts, and reaction conditions in order to provide a thorough description of embodiments of the disclosure. However, a person of ordinary skill in the art will understand that the embodiments of the disclosure may be practiced without employing these specific details. Indeed, the embodiments of the disclosure may be practiced in conjunction with conventional processes employed in the industry. In addition, the description provided below does not form a complete process flow for producing the GLYN. Only those process acts necessary to understand the embodiments of the disclosure are described in detail below. Additional acts may be performed by conventional techniques. Also note, any drawings accompanying the application are for illustrative purposes only, and are thus not drawn to scale. Additionally, elements common between figures may retain the same numerical designation.

The glycerol of the diluted glycerol is nitrated in a first reaction channel of the microfluidic reactor to form the nitrated glycerol compound and an intramolecular ring closure is conducted in a second reaction channel of the microfluidic reactor and on the nitrated glycerol compound according to the following reaction scheme:

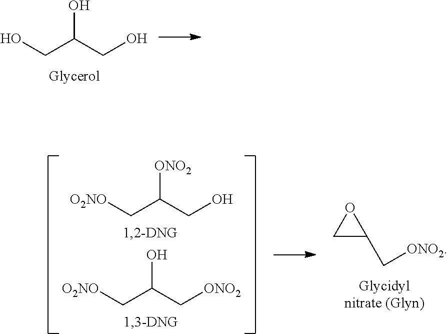

The nitration reaction and the intramolecular condensation reaction may be conducted in series in the microfluidic reactor. The glycerol is nitrated with the nitrating agent, such as nitric acid (HNO3), to form the nitrated glycerol compound, such as a dinitroglycerol (DNG) compound. The DNG compound may include, but is not limited to, 1,2-dinitroglycerol, 1,3-dinitroglycerol, or combinations thereof. While specific examples herein disclose using nitric acid as the nitrating agent, other nitrating agents may be used, such as a combination of sulfuric acid and nitric acid, acetyl nitrate, a nitronium ion salt, etc.

Other nitrated glycerol compounds, such as a mononitroglycerol (MNG) compound or trinitroglycerol (NG), may be formed as byproducts during the nitration reaction. To achieve a high yield of GLYN, the amount of DNG compound produced during the nitration reaction may be maximized (e.g., the DNG compound may be a major reaction product of the nitration reaction), with trace amounts or substantially no MNG compound or NG produced. The MNG compound, if present, reduces the purity of the resulting GLYN and affects subsequent polymerization of the GLYN. The NG, if present, increases the risk of hazards associated with the nitration reaction, as well as reduces the purity of the resulting GLYN. Reaction temperature, nitric acid concentration, ratio of nitric acid:glycerol, and residence time may be adjusted to produce the DNG compound as the major reaction product of the nitration reaction. If, however, the MNG compound and/or NG are produced, these byproducts may be removed to increase the purity of the DNG compound.

To form the diluted glycerol, neat glycerol, having a purity of greater than or equal to 99.0%, is diluted with a solvent before conducting the nitration reaction. The solvent may be miscible with the neat glycerol and relatively inert (e.g., non-reactive) with the neat glycerol. By way of example only, water may be added to the neat glycerol. The neat glycerol may be diluted with water (e.g., deionized water) to achieve a decreased viscosity suitable for use in the microfluidic reactor. The decreased viscosity may enable the diluted glycerol to be easily transported through the microfluidic reactor. The diluted glycerol does not have a significant effect on the relative amounts of the reaction products of the nitration reaction as compared to the relative amounts of the reaction products formed when using neat glycerol. The viscosity of the diluted glycerol may be selected depending on the operating conditions of the microfluidic reactor, such as on the reaction temperature or reaction pressure of the microfluidic reactor during the nitration reaction.

Adding water to the neat glycerol may form an aqueous glycerol solution that includes from about 30% by weight (wt %) to about 95 wt % glycerol, such as from about 30 wt % to about 90 wt % glycerol, from about 35 wt % to about 90 wt % glycerol, from about 40 wt % to about 90 wt % glycerol, from about 45 wt % to about 90 wt % glycerol, from about 50 wt % to about 90 wt % glycerol, from about 55 wt % to about 90 wt % glycerol, from about 60 wt % to about 90 wt % glycerol, from about 65 wt % to about 90 wt % glycerol, from about 70 wt % to about 90 wt % glycerol, from about 75 wt % to about 90 wt % glycerol, from about 75 wt % to about 85 wt % glycerol, from about 75 wt % to about 80 wt % glycerol, from about 80 wt % to about 85 wt % glycerol, or from about 80 wt % to about 95 wt % glycerol. In some embodiments, the aqueous glycerol solution includes from about 75 wt % to about 85 wt % glycerol. In other embodiments, the aqueous glycerol solution includes from about 80 wt % to about 90 wt % glycerol.

The viscosity of the diluted glycerol may be less than or equal to about 300 cP at 20° C., such as less than or equal to about 200 cP at 20° C., less than or equal to about 150 cP at 20° C., or less than or equal to about 100 cP at 20° C. For instance, diluted glycerol including about 30 wt % glycerol may exhibit a viscosity of about 2.5 cP at 20° C., or diluted glycerol including about 90 wt % glycerol may exhibit a viscosity of about 210 cP at 20° C. In contrast, neat glycerol has a viscosity of about 1,400 cP at 20° C. and a viscosity of about 612 cP at 30° C., which may cause clogging of the microfluidic reactor. Maximum pressure levels within the microfluidic reactor may be met or exceeded when neat glycerol is used to produce GLYN. By reducing the viscosity, flow of the diluted glycerol through pumps and channels of the microfluidic reactor may be improved, reducing hazards associated with clogging of the microfluidic reactor. By using the diluted glycerol in the nitration reaction, peristaltic pumps, which are able to move lower viscosity fluids and reduce overall pressure inside the microfluidic reactor, may be used to transport the reagents throughout the microfluidic reactor.

It was unexpected and surprising that using the diluted glycerol provided a significant reduction in viscosity without decreasing the relative yield of the nitrated glycerol compound (e.g., DNG compound) compared to using neat glycerol. With conventional processes for forming GLYN, it was believed that the addition of water to neat glycerol would greatly reduce the yield of the DNG compound produced, due to the weaker nitrating power of the nitric agent. It was also believed that the concentration of nitric acid would need to be increased to produce the DNG compound. However, with the GLYN process according to embodiments of the disclosure, it was surprisingly found that a significant amount of water may be added to the neat glycerol without changing the concentration (e.g., grade) of nitric acid used and without affecting the yield of the DNG compound. Without being bound by any theory, it is believed that inter- and intra-molecular hydrogen bonds form between the glycerol and water, decreasing the viscosity of the diluted glycerol relative to the viscosity of neat glycerol.

The nitric acid used as the nitrating agent may be concentrated nitric acid (98%) or a nitric acid solution. The nitric acid solution may include the nitric acid and a solvent, such as water or dichloromethane (DCM). A ratio of the solvent to the nitric acid (solvent:nitric acid) may range from about 0.01:1 to about 1:1. In some embodiments, concentrated nitric acid is used as the nitrating agent. In other embodiments, a nitric acid solution including about 90 wt % nitric acid and about 10 wt % water is used.

An excess of the nitric acid may be used relative to the glycerol in the diluted glycerol. A ratio of the nitric acid to diluted glycerol (nitric acid:diluted glycerol) may range from about 2.0:1.0 to about 10.0:1.0, such as from about 2.0:1.0 to about 10.0:1.0, 3.0:1.0 to about 10.0:1, from about 4.0:1.0 to about 10.0:1.0, from about 4.0:1.0 to about 8.0:1.0, from about 3.0:1.0 to about 8.0:1.0, from about 3.0:1.0 to about 6.0:1.0, from about 3.0:1.0 to about 5.0:1.0, or from about 3.5:1.0 to about 4.5:1.0. In some embodiments, the ratio of nitric acid to diluted glycerol is about 4.0:1.0. The nitric acid:diluted glycerol ratio is based on flow rates of each reagent.

The diluted glycerol and nitric acid may be reacted in the first reaction channel of the microfluidic reactor for an amount of time sufficient to form the nitrated glycerol compound. The time may range from about 5 seconds to about 10 minutes, such as from about 5 seconds to about 5 minutes, from about 5 seconds to about 2 minutes (120 seconds), from about 5 seconds to about 1.5 minutes (90 seconds), from about 10 seconds to about 1.5 minutes (90 seconds), from about 10 seconds to about 1 minute (60 seconds), from about 15 seconds to about 1.5 minutes (90 seconds), or from about 15 seconds to about 1 minute (60 seconds). In some embodiments, the nitration reaction time (e.g., residence time) is about 60 seconds. In other embodiments, the nitration reaction time (e.g., residence time) is about 120 seconds.

The diluted glycerol and nitric acid may be reacted at a temperature ranging from about 15° C. to about 55° C., such as from about 15° C. to about 40° C., from about 15° C. to about 30° C., from about 15° C. to about 25° C., from about 15° C. to about 20° C., from about 20° C. to about 30° C., from about 20° C. to about 35° C., from about 20° C. to about 50° C., from about 20° C. to about 40° C., or from about 20° C. to about 25° C. In some embodiments, the nitration reaction is conducted at a reaction temperature of about 20° C. In other embodiments, the nitration reaction is conducted at a reaction temperature of about 25° C. In yet other embodiments, the nitration reaction is conducted at a reaction temperature of about 30° C. In still yet other embodiments, the nitration reaction is conducted at a reaction temperature of about 35° C. By controlling the temperature of the nitration reaction, the DNG compound may be produced at a higher purity by decreasing side reactions that produce the MNG compound or NG. Controlling the temperature of the nitration reaction may also affect relative amounts of the DNG compound produced, such as predominantly forming 1,2-DNG or predominantly forming 1,3-DNG.

The diluted glycerol and nitric acid may be introduced into the first reaction channel of the microfluidic reactor at a flow rate sufficient for the diluted glycerol and nitric acid to react to substantial completion in the reaction channel. The flow rate may be selected to achieve the desired nitric acid:glycerol ratio and residence time. The flow rate may, for example, be within a range of from about 0.05 ml/minute to about 10.00 ml/minute, from about 0.05 ml/minute to about 8.00 ml/minute, from about 0.05 ml/minute to about 7.50 ml/minute, from about 0.05 ml/minute to about 7.00 ml/minute, from about 0.10 ml/minute to about 6.00 ml/minute, from about 0.10 ml/minute to about 5.50 ml/minute, from about 0.10 ml/minute to about 5.00 ml/minute, from about 0.10 ml/minute to about 4.50 ml/minute, from about 0.10 ml/minute to about 4.00 ml/minute, from about 0.10 ml/minute to about 3.50 ml/minute, from about 0.10 ml/minute to about 3.00 ml/minute, from about 0.10 ml/minute to about 2.50 ml/minute, from about 0.10 ml/minute to about 2.00 ml/minute, from about 0.10 ml/minute to about 1.50 ml/minute, from about 0.10 ml/minute to about 1.25 ml/minute, from about 0.125 ml/minute to about 1.25 ml/minute, from about 0.15 ml/minute to about 1.25 ml/minute, from about 0.25 ml/minute to about 1.25 ml/minute, or from about 0.75 ml/minute to about 1.25 ml/minute. In some embodiments, the flow rate is 0.152 ml/minute. In other embodiments, the flow rate is 0.76 ml/minute. In yet other embodiments, the flow rate is 0.38 ml/minute. The flow rate of the glycerol and the nitric acid may be the same or may be different.

The residence time of the reaction products of the nitration reaction may range from about 0.25 minutes to about 30.0 minutes, such as from about 0.25 minutes to about 25.0 minutes, from about 5.0 minutes to about 22.0 minutes, from about 10.0 minutes to about 22.0 minutes, from about 15.0 minutes to about 22.0 minutes, or from about 20.0 minutes to about 22.0 minutes. In some embodiments, the residence time is 21.0 minutes. The diluted glycerol and nitric acid may substantially completely react in about 20 minutes, such as between about 20 minutes and about 30 minutes. The short reaction time for the nitration reaction was unexpected relative to the reaction times of conventional glycerol nitration processes.

The nitration reaction may produce the DNG compound at a conversion of greater than or equal to about 50 mole percent (mol %), such as from about 50 mol % to about 85 mol %, from about 55 mol % to about 80 mol %, from about 50 mol % to about 75 mol %, from about 50 mol % to about 70 mol %, from about 60 mol % to about 80 mol %, from about 65 mol % to about 80 mol %, from about 65 mol % to about 75 mol %, or from about 70 mol % to about 80 mol %. In some embodiments, the DNG compound is produced at greater than or equal to about 70 mol %.

The nitration reaction produces a DNG solution that includes the DNG compound, excess (e.g., unreacted) nitric acid, water-soluble byproducts, NG, and water. The excess nitric acid, the water-soluble byproducts, and the water may be removed by liquid:liquid separation techniques before conducting the intramolecular ring closure reaction. An organic solvent, such as DCM, may be added to the DNG solution, producing a biphasic DNG solution. The organic solvent may be immiscible with the water of the DNG solution. A sufficient volume of the organic solvent may be added to produce an organic phase and an aqueous phase. Additional water may optionally be added to achieve the biphasic DNG solution. If, however, the DNG solution includes a sufficient amount of water from the diluted glycerol to produce the biphasic DNG solution, additional water may not be needed. The DNG compound is extracted into the organic phase of the biphasic DNG solution, and the nitric acid and water-soluble byproducts are extracted into the aqueous phase of the biphasic DNG solution. From about 40% to about 70% of the DNG compound may be extracted into the organic phase (e.g., into the DCM). The organic and aqueous phases may be contacted for a sufficient residence time for the DNG compound to extract into the organic phase, such as from about 5 seconds to about 5 minutes or from about 5 seconds to about 2 minutes. Additional DNG compound may be recovered by conducting one or more additional extractions.

The organic and aqueous phases of the biphasic DNG solution may be separated using a liquid:liquid phase separator. The liquid:liquid phase separator may, for example, include a membrane-based liquid:liquid phase separator, such as those available from Zaiput Flow Technologies (Waltham, Mass.). However, other conventional liquid:liquid phase separators may be used. The liquid:liquid phase separator may have an internal volume of about 0.5 ml and may be configured to handle a flow rate of from about 10 ml/min to about 100 ml/min. The liquid:liquid phase separator may also include a hydrophobic membrane. The biphasic DNG solution is flowed through the liquid:liquid phase separator, which is configured in-line with the microfluidic reactor. By separating the organic and aqueous phases, at least a portion of the nitric acid may be separated from the DNG compound. Therefore, a lower amount of base may be used during the intramolecular ring closure reaction. Reducing the amount of base needed may increase a total output by GLYN by the system. In addition, separating the aqueous phase separates water-soluble products, such as MNG, from the GLYN. If present, the water-soluble products may negatively affect the polymerization of GLYN to PGN. Salt formation within the microfluidic reactor may also be reduced or eliminated by removing the nitric acid following the nitration reaction, which reduces or prevents clogging of the microfluidic reactor. Reducing the salt formation also removes the necessity of keeping the salts solubilized. In addition, exotherms associated with neutralization of the nitric acid may be avoided. The removal of the nitric acid before conducting the intramolecular ring closure reaction also greatly reduces safety hazards and maintenance costs associated with clogging.

After separating the organic and aqueous phases, the intramolecular ring closure reaction is conducted on the organic phase containing the DNG compound. Alternatively, the DNG compound may be recovered from the organic phase before conducting the intramolecular ring closure reaction. The DNG compound contained in the organic phase is exposed to the base in the second reaction channel of the microfluidic reactor to induce the intramolecular ring closure and form the GLYN. The base may increase the pH and induce the intramolecular ring closure reaction. The GLYN may be formed in a GLYN solution. In addition to producing GLYN, the intramolecular ring closure reaction may form neutralization salts. A concentration of the DNG compound in the organic phase may range from about 0.2 M to about 3.0 M, such as from about 0.4 M to about 3.0 M, from about 0.4 M to about 2.0 M, from about 0.4 M to about 1.5 M, from about 1.5 M to about 3.0 M, from about 2.0 M to about 3.0 M, from about 1.5 M to about 2.5 M, from about 1.5 M to about 2.0 M, or from about 2.0 M to about 2.5 M. The base may be introduced into the organic phase containing the DNG compound. The base may include, but is not limited to, potassium hydroxide (KOH). The potassium hydroxide may be added as an aqueous solution of potassium hydroxide, which may have a lower viscosity than a similar concentration of sodium hydroxide. A concentration of the potassium hydroxide may range from about 1.5 M to about 10.0 M, such as from about 1.5 M to about 8.0 M, from about 3.0 M to about 10.0 M, from about 5.0 M to about 8.0 M, from about 6.0 M to about 9.0 M, or from about 7.0 M to about 8.0 M. In some embodiments, the concentration of potassium hydroxide in the aqueous solution of potassium hydroxide is about 7.2 M.

The conversion from glycerol to GLYN during the intramolecular ring closure reaction may be from about 50 mol % to about 90 mol %, such as from about 50 mol % to about 85 mol %, from about 50 mol % to about 80 mol %, from about 55 mol % to about 90 mol %, from about 60 mol % to about 90 mol %, from about 70 mol % to about 90 mol %, or from about 70 mol % to about 80 mol %. In some embodiments, the conversion from glycerol to GLYN is about 58 mol %.

Using potassium hydroxide as the base may produce potassium salts, which exhibit increased solubility in water compared to sodium salts. Therefore, neutralization salts (e.g., potassium salts, potassium nitrate) produced after the caustic treatment may exhibit increased solubility in an aqueous solution compared to the solubility of sodium salts (e.g., sodium nitrate). While sodium hydroxide may be used in conventional batch processes because of the lower amount of heat evolved when neutralizing nitric acid compared to potassium hydroxide, the superior heat transfer in the microfluidic reactor may compensate for this increase. Using potassium hydroxide as the base to induce the intramolecular ring closure reaction also showed significantly lower clogging risk compared to using sodium hydroxide, and did not produce a measurable exotherm in the microfluidic reactor during acid neutralization. With the higher molecular mass of potassium hydroxide, a greater amount of potassium hydroxide is used to neutralize the nitric acid compared to sodium hydroxide, which also increases the amount of heat produced.

The base may be present in excess. For example, a volume ratio of the DNG compound in solution (i.e., the organic phase containing the DNG compound) to the base may range from about 1.0:6.0 to about 2.0:1.0. A mole ratio of the DNG compound to the base may range from about 1:50 to about 1:4.

The DNG compound and the base may be reacted in the microfluidic reactor at a temperature of from about 20° C. to about 60° C., such as from about 20° C. to about 55° C., from about 25° C. to about 55° C., from about 30° C. to about 55° C., from about 35° C. to about 55° C., from about 40° C. to about 55° C., from about 45° C. to about 55° C., from about 50° C. to about 55° C., from about 30° C. to about 60° C., from about 35° C. to about 60° C., from about 40° C. to about 60° C., from about 45° C. to about 60° C., from about 50° C. to about 60° C., or from about 55° C. to about 60° C. In some embodiments, the temperature is about 50° C. In other embodiments, the temperature is about 55° C. Controlling the temperature of the intramolecular condensation reaction may produce the GLYN at a higher purity by decreasing side reactions.

The DNG compound in the organic phase and the base may be introduced into the second reaction channel of the microfluidic reactor at a flow rate within a range of from about 0.1 ml/minute to about 10.0 ml/minute, from about 0.5 ml/minute to about 10.0 ml/minute, from about 1.0 ml/minute to about 10.0 ml/minute, from about 1.0 ml/minute to about 9.0 ml/minute, from about 1.0 ml/minute to about 8.0 ml/minute, from about 1.0 ml/minute to about 7.0 ml/minute, from about 1.0 ml/minute to about 6.0 ml/minute, or from about 1.0 ml/minute to about 5.0 ml/minute. In some embodiments, the flow rate is about 5.0 ml/minute. In other embodiments, the flow rate is about 6.0 ml/minute.

The residence time of the intramolecular condensation reaction may range from about 0.5 minutes to about 30.0 minutes, such as from about 2.0 minutes to about 25.0 minutes, from about 2.0 minutes to about 25.0 minutes, from about 5.0 minutes to about 25.0 minutes, from about 10.0 minutes to about 25.0 minutes, from about 15.0 minutes to about 25.0 minutes, from about 20.0 minutes to about 25.0 minutes, from about 1 minute to about 15 minutes, from about 2 minutes to about 10 minutes, from about 4 minutes to about 8 minutes, or from about 4 minutes to about 6 minutes. In some embodiments, the residence time is about 1.2 minutes. In other embodiments, the residence time is about 0.7 minutes. In comparison, converting the DNG compound to GLYN by conventional batch processes takes between 2 hours and 3 hours.

To separate the GLYN from other products in the GLYN solution, additional organic solvent, such as DCM, may be added to produce a biphasic GLYN solution. A sufficient volume of the organic solvent may be added to produce an organic phase and an aqueous phase of the GLYN solution. Since the DNG compound is contained in DCM following the nitration reaction and extraction, a sufficient volume of DCM may, however, already be present in the GLYN solution. Additional water may optionally be added to achieve the biphasic GLYN solution. The organic and aqueous phases may be separated using liquid:liquid phase separators, such as those disclosed above, that are configured in-line with the microfluidic reactor. The GLYN is extracted into the organic phase of the biphasic GLYN solution. The MNG, potassium hydroxide, potassium salts, and other water-soluble byproducts are extracted into the aqueous phase of the biphasic GLYN solution. By separating the organic and aqueous phases, the MNG, potassium hydroxide, potassium salts, and other water-soluble byproducts, which can affect polymerization of GLYN, are removed. The GLYN may be extracted into the DCM at a residence time of less than or equal to about 60 seconds, such as between about 1 second and about 60 seconds, between about 5 seconds and about 50 seconds, between about 5 seconds and about 40 seconds, between about 5 seconds and about 30 seconds, or between about 5 seconds and about 20 seconds. The reactions and the separations (e.g., extractions) may be conducted in a single system, enabling the GLYN to be produced continuously without having to extract the GLYN separately.

Since the GLYN is in the organic phase of the biphasic GLYN solution, the GLYN may be easily recovered. The extraction of the GLYN into the DCM also reduces or eliminates the purification and work-up following the intramolecular condensation reaction. However, the recovered GLYN may, optionally, be subjected to further purification before polymerizing the GLYN to form PGN. The purification of the GLYN may be conducted by conventional techniques. The PGN may be produced from the GLYN by conventional techniques.

Experiments conducted for the production of GLYN according to embodiments of the disclosure are included in Examples 1-11 below. Reaction products of the nitration reaction and the intramolecular ring closure reaction were analyzed by conventional techniques, such as by HPLC, NMR, and/or by GC/MS, and were tested in duplicate or in triplicate.

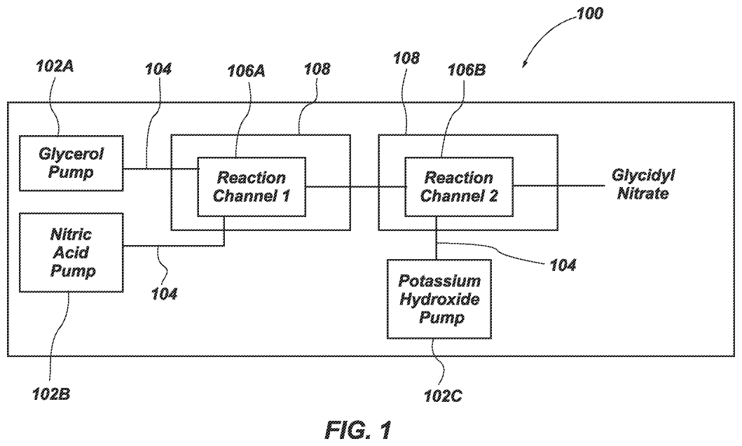

A system including a microfluidic reactor and liquid:liquid phase separators is also disclosed. The microfluidic reactor includes multiple reactor plates that define channels in which the nitration reaction and the intramolecular ring closure reaction are conducted. The liquid:liquid phase separators are configured in-line with the reactor plates of the microfluidic reactor. The microfluidic reactor is configured for a capacity of up to about 10 ml/min. While dimensions of components of the microfluidic reactor are described herein, the components of the microfluidic reactor may have larger dimensions if higher amounts of GLYN are desired. In other words, the dimensions of the components of the microfluidic reactor may be increased if the amount of GLYN to be produced is scaled up. As shown in FIG. 1, the nitration reaction and the intramolecular ring closure reaction are conducted in a microfluidic reactor 100 that includes pumps 102, tubing 104, at least one reaction channel 106 (e.g., at least one residence tube), and at least one temperature control system 108. The microfluidic reactor 100 is configured to provide control of reagent flow rates, reagent mixing, reaction temperature, and residence time. The pumps 102 may be selected to provide a flow rate of from about 10 ml/min to about 100 ml/min. The components of the microfluidic reactor 100 are configured to be compatible with the reagents, optional solvents, and other process conditions of the reactions. Since the glycerol is reacted under acidic conditions and the DNG compound is reacted under basic conditions, the components of the microfluidic reactor 100 are substantially resistant to acidic and basic conditions. The reagents are continuously introduced into the reaction channels 106 of the microfluidic reactor 100 using the pumps 102 and the reactions occur in the reaction channels 106 at a desired reaction temperature. The reaction temperature is controlled by the temperature control systems 108, which also dissipate heat generated by the exothermic reactions.

The microfluidic reactor 100 may, for example, be configured as a glass chip or a glass plate having one or more reaction channels 106 etched in the glass. Internal dimensions of the reaction channels 106 may be between about 1 μm and about 1000 μm. The reaction volume of the microfluidic reactor 100 may depend on the dimensions (e.g., inner diameter, length) of the reaction channels 106 in which the nitration reaction and the intramolecular ring closure reaction are conducted. The reaction channels 106 of the microfluidic reactor 100 have a sufficiently high surface area to bulk ratio to enable quick and efficient heat dissipation.

The pumps 102 are configured to introduce the reagents into the microfluidic reactor 100 at a desired flow rate, such as at a constant flow rate. Pumps 102 of varying sizes may be used to achieve the desired flow rate, such as up to about 100 ml/min, and reagent feed ratio. The diluted glycerol and nitric acid are introduced into the microfluidic reactor 100 through inlets (not shown) and combined with mixing in the first reaction channel 106A. The diluted glycerol and nitric acid may be introduced into the system by separate pumps 102A, 102B. Alternatively, glycerol, water, and nitric acid may be introduced into the system by separate pumps 102, with the glycerol and water combining to form the diluted glycerol. The potassium hydroxide and dichloromethane are introduced into the microfluidic reactor 100 through inlets (not shown) and combined with the DNG compound in the second reaction channel 106B. The potassium hydroxide may be introduced into the system by a pump 102C. The potassium hydroxide and dichloromethane may be introduced into the system by separate pumps 102. The reaction of the diluted glycerol and nitric acid and the DNG compound and potassium hydroxide is based on the flow rate of the reagents through the microfluidic reactor 100 and the length of the first and second reaction channels 106A, 106B. The flow rate of each of the reagents may be the same or may be different. Alternatively, the desired reagent feed ratio of each of the reagents may be achieved by dilution. Depending on the amount of GLYN to be produced, the pumps 102 may be syringe pumps or other conventional pumps, such as peristaltic pumps. The pumps 102 may include check valves to prevent back flow and auto shut-offs when approaching a set pressure limit.

The tubing 104 is configured to connect the components of the microfluidic reactor 100 to one another, such as to connect the pumps 102 to the first and second reaction channels 106A, 106B. The tubing 104 may, for example, be resistant to the acidic conditions of the nitration reaction and resistant to the basic conditions of the intramolecular condensation reaction. A material of the tubing 104 may, for example, be a polymeric material, a glass material, or a ceramic material. The tubing 104 may transport the glycerol, nitric acid, and potassium hydroxide from the pumps 102 to the first and second reaction channels 106A, 106B. In some embodiments, the tubing 104 is polytetrafluoroethylene (PTFE) tubing. The tubing 104 may have an inner diameter of less than or equal to about 1 mm.

The reagents enter the first and second reaction channels 106A, 106B and are combined by mixing, such as by laminar flow mixing, diffusional mixing, etc. The reagents may be combined within a range from milliseconds to seconds due to the small reaction volume of the first and second reaction channels 106A, 106B.

The microfluidic reactor 100 may include one or more reaction channels 106 configured to react the reagents (e.g., diluted glycerol and nitric acid, DNG compound and base) to form the GLYN. The reaction channels 106 are substantially resistant to the reagents and solvents used in the reactions. The material of the reaction channels 106 may be resistant to the acidic conditions of the nitration reaction and the basic conditions of the intramolecular condensation reaction. The reaction channels 106 may, for example, be formed of a polymeric material, a glass material, or a ceramic material. In some embodiments, the reaction channel 106 is formed from PTFE. In other embodiments, the reaction channel 106 is formed from glass. The reaction channels 106 may have an inner diameter of less than or equal to about 1 mm, such as from about 1 μm to about 1.0 mm (1000 μm), from about 1 μm to about 500 μm, from about 1 μm to about 400 μm, from about 1 μm to about 300 μm, from about 1 μm to about 200 μm, from about 1 μm to about 100 μm, from about 10 μm to about 100 μm, from about 0.1 mm (100 μm) to about 1.0 mm (1000 μm), from about 0.2 mm to about 0.9 mm, from about 0.3 mm to about 0.8 mm, from about 0.4 mm to about 0.8 mm, from about 0.5 mm to about 0.8 mm, from about 0.6 mm to about 0.8 mm, or from about 0.7 mm to about 0.8 mm. In some embodiments, the reaction channels 106 have an inner diameter of from about 0.7 mm to about 0.8 mm. A length of the reaction channels 106 may be sufficient for substantially all of the reagents to react within the reaction channels 106 and may be selected depending on the amount of GLYN to be produced and the reaction volume of the microfluidic reactor 100. The length of the reaction channels 106 may range from about 30 cm to about 400 cm.

A reaction volume of the microfluidic reactor may be less than or equal to about 40 ml, such as from about 1 μl to about 20 ml, from about 1 μl to about 100 μl, from about 1 μl to about 1000 μl, from about 5 μl to about 100 μl, from about 10 μl to about 90 μl, from about 10 μl to about 80 μl, from about 10 μl to about 50 μl, from about 100 μl to about 1000 μl, from about 1 ml to about 20 ml, from about 5 ml to about 20 ml, from about 5 ml to about 15 ml, from about 5 ml to about 10 ml, from about 10 ml to about 20 ml, from about 15 ml to about 20 ml, or from about 10 ml to about 20 ml.

The temperature control system 108 is configured to maintain the reagents in the reaction channels 106 at a desired temperature during the nitration and intramolecular condensation reactions. The temperature control system 108 may be configured to maintain the reaction channels 106 at a constant temperature during the nitration and intramolecular condensation reactions. The temperature control system 108 is also configured to dissipate heat generated by the exothermic nitration and intramolecular condensation reactions. The temperature control system 108 may, for example, be a temperature bath in which the reaction channels 106 are immersed. Alternatively, the temperature control system 108 may include a jacketed cooling loop. The microfluidic reactor 100 has a high surface area to bulk ratio, enabling quick and efficient dissipation of the heat generated by the exothermic reactions. A higher degree of temperature control may, therefore, be achieved relative to conventional processes of producing GLYN.

After conducting the intramolecular condensation reaction, the GLYN exits the second reaction channel 106B and is collected. The GLYN is extracted from the GLYN solution as described above and recovered. The GLYN formed according to embodiments of the disclosure may subsequently be converted to PGN. The PGN may be used, for example, as an ingredient in a propellant, an insensitive explosive, a gas generant, etc. The PGN may be used in a rocket motor, such as a solid rocket motor, or in a warhead.

The microfluidic reactor 100 may be a commercially available continuous flow reactor, such as ADVANCED-FLOW® from Dow Corning (Corning, NY), LABTRIX® (reaction volume 1-19.5 μl), PROTRIX® (reaction volume 1-13.5 ml), GRAMFLOW® (reaction volume up to 1 ml), KILOFLOW® (reaction volume 0.8-18 ml), or PLANTRIX® (reaction volume 100 ml-4 L), from Chemtrix BV (Echt, the Netherlands). However, the microfluidic reactor 100 may be obtained from other commercial sources.

The following examples serve to explain embodiments of the disclosure in more detail. These examples are not to be construed as being exhaustive or exclusive as to the scope of this disclosure.

### EXAMPLES

### Example 1

### Microfluidic Reactor Configuration

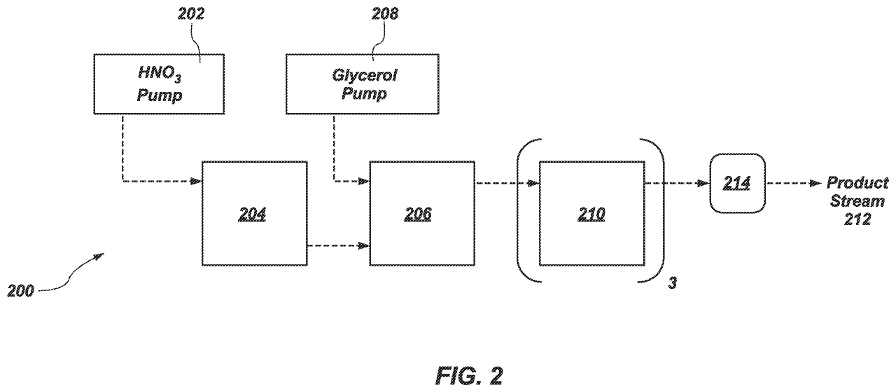

Referring to FIG. 2, a configuration 200 of an ADVANCED-FLOW® Reactor (AFR) from Dow Corning is shown. The AFR included a nitric acid pump 202 configured to pump nitric acid through a series of plates 204, 206, 210. Nitric acid supplied by the nitric acid pump 202 entered into a temperature equilibration plate 204. Temperature equilibrated nitric acid from the temperature equilibration plate 204 then entered into a mixing plate 206. A glycerol pump 208 was used to supply glycerol to the mixing plate 206. The nitric acid and the glycerol were mixed in the mixing plate 206 and then flowed through three residence plates 210 to produce a product stream 212. To provide temperature control, each of the plates (e.g., the temperature equilibration plate 204, the mixing plate 206, and the three residence plates 210) was jacketed in a temperature regulated 20% glycerol solution. The temperature of the 20% glycerol solution was regulated with a recirculating heater/chiller. The AFR was configured with a back pressure regulator 214, such that the product stream 212 flowed through the back pressure regulator 214 after exiting the series of plates.

### Example 2

### Nitration Process Conditions

Glycerol and nitric acid were injected into the AFR described in Example 1. The glycerol and nitric acid were reacted at various process conditions and the reaction products in the product stream were analyzed to determine relative percentages of reaction products, including the two isomers of MNG, the two isomers of DNG, and NG. The effects of reaction temperature, nitric acid to glycerol ratio, residence time, and back flow pressure on the relative percentages of the reaction products were evaluated. The reaction products were analyzed by conventional techniques, such as by HPLC, NMR, and/or by GC/MS, and were tested in duplicate or in triplicate. Table 1 includes the process variables (reaction temperature (e.g., set temperature), back flow pressure, nitric acid flow rate, glycerol flow rate, ratio of nitric acid:glycerol, total input flow rate, and residence time) for each of Samples 1-16. The flow rates were selected to achieve the desired ratio of nitric acid:glycerol and residence times. The residence times included in Table 1 were calculated based on the AFR volume and total input flow rate. Actual residence times were approximately 5-10% longer than shown in Table 1.

TABLE 1
Sample Process Variables

| | | | | | | | Calculated |
|---|---|---|---|---|---|---|---|
| | Set Temp | Back flow | HNO₃ Flow | Glycerol Flow | | Flow Rate | Residence |
| Sample | ° C. | pressure (Bar) | Rate (mL/min) | Rate (mL/min) | HNO₃:Glycerol | (mL/min) | Time (Sec) |
| 1 | 20 | 1.0 | 1.600 | 0.400 | 4:1 | 2.000 | 60 |
| 2 | 20 | 5.0 | 1.778 | 0.222 | 8:1 | 2.000 | 60 |
| 3 | 20 | 5.0 | 6.000 | 1.500 | 4:1 | 7.500 | 16 |
| 4 | 20 | 5.0 | 6.665 | 0.833 | 8:1 | 7.498 | 16 |
| 5 | 35 | 5.0 | 1.600 | 0.400 | 4:1 | 2.000 | 60 |
| 6 | 35 | 5.0 | 1.778 | 0.222 | 8:1 | 2.000 | 60 |
| 7 | 35 | 1.0 | 6.000 | 1.500 | 4:1 | 7.500 | 16 |
| 8 | 35 | 1.0 | 1.778 | 0.222 | 8:1 | 2.000 | 60 |
| 9 | 20 | 5.0 | 1.778 | 0.222 | 8:1 | 2.000 | 60 |
| 10 | 20 | 5.0 | 6.000 | 1.500 | 4:1 | 7.500 | 16 |
| 11 | 20 | 1.0 | 6.665 | 0.833 | 8:1 | 7.498 | 16 |
| 12 | 35 | 1.0 | 1.600 | 0.400 | 4:1 | 2.000 | 60 |
| 13 | 35 | 1.0 | 6.665 | 0.833 | 8:1 | 7.498 | 16 |
| 14 | 35 | 5.0 | 6.000 | 1.500 | 4:1 | 7.500 | 16 |
| 15 | 35 | 5.0 | 6.665 | 0.833 | 8:1 | 7.498 | 16 |
| 16 | 20 | 5.0 | 1.600 | 0.400 | 4:1 | 2.000 | 60 |

Table 2 includes the resulting relative percentages of the reaction products (the two isomers of MNG, the two isomers of DNG, and NG) for each of the Samples 1-16.

TABLE 2
Sample Reaction Product Results

| | 2-Mono | l-Mono | 1,3- | 1,2- | | |
|---|---|---|---|---|---|---|
| Sample | NG | NG | DiNG | DiNG | NG | Glycerol |
| 1A | 2.0% | 33.4% | 48.4% | 6.7% | 3.6% | 5.8% |
| 1B | 2.0% | 33.3% | 49.1% | 6.8% | 3.8% | 4.9% |
| 2A | 0.1% | 1.6% | 42.6% | 9.0% | 46.6% | NM |
| 2B | 0.2% | 2.6% | 43.8% | 9.9% | 43.5% | 0.0% |
| 3A | 2.9% | 48.2% | 41.8% | 5.6% | 1.5% | NM |
| 3B | 2.9% | 48.6% | 41.4% | 5.6% | 1.5% | NM |
| 3C | 1.7% | 30.9% | 56.4% | 7.3% | 3.8% | NM |
| 4A | 0.1% | 2.5% | 62.1% | 6.7% | 28.6% | NM |
| 4B | 0.0% | 2.2% | 50.6% | 7.2% | 40.0% | NM |
| 4C | 0.2% | 2.0% | 51.3% | 8.8% | 37.7% | NM |
| 5A | 0.4% | 7.0% | 69.4% | 8.3% | 15.0% | NM |
| 5B | 1.0% | 16.9% | 61.7% | 8.4% | 9.1% | 3.0% |
| 6A | 0.2% | 1.6% | 39.9% | 10.2% | 48.2% | NM |
| 6B | 0.2% | 2.7% | 46.8% | 11.7% | 38.6% | 0.0% |
| 7A | 0.8% | 15.0% | 67.2% | 8.0% | 9.0% | NM |
| 7B | 2.4% | 37.7% | 47.4% | 7.2% | 3.2% | 2.1% |
| 8A | 0.2% | 1.5% | 36.3% | 8.9% | 53.1% | NM |
| 8B | 0.2% | 2.6% | 44.7% | 11.3% | 41.2% | 0.0% |
| 9A | 0.3% | 2.9% | 47.0% | 10.5% | 37.4% | 2.0% |
| 10A | 3.7% | 57.7% | 25.3% | 3.8% | 1.0% | 8.5% |
| 11A | 0.1% | 3.6% | 66.6% | 6.4% | 23.0% | 0.4% |
| 11B | 0.1% | 3.3% | 59.3% | 5.7% | 31.6% | 0.0% |
| 12A | 0.4% | 7.8% | 71.3% | 8.2% | 12.3% | NM |
| 12B | 1.0% | 16.8% | 62.3% | 8.4% | 7.3% | 4.2% |
| 13A | 0.3% | 3.8% | 59.1% | 11.0% | 25.6% | 0.4% |
| 14A | 0.6% | 12.8% | 68.0% | 8.1% | 10.5% | NM |
| 14B | 2.4% | 37.2% | 46.2% | 7.0% | 3.3% | 3.8% |
| 15A | 0.0% | 1.9% | 46.0% | 9.8% | 42.3% | NM |
| 15B | 0.3% | 3.5% | 56.0% | 10.7% | 29.6% | 0.0% |
| 16A | 1.1% | 20.6% | 63.7% | 8.2% | 6.5% | NM |

1. The alphabetical notation in the sample number column denotes replicates (i.i e., A replicate 1, B replicate 2, and so on.)
2. NM; Not measured

The results of Samples 2 and 4 showed that at a reaction temperature of 20° C., a higher ratio of nitric acid to glycerol resulted in a lower relative amount of MNG in the product stream. However, this effect was less pronounced at elevated temperatures, such as at a temperature greater than 20° C. and less than or equal to 35° C. The results of Samples 5 and 12 showed that at a nitric acid to glycerol ratio of 4:1, the amount of MNG in the product stream was minimized with a relatively longer residence time and a relatively higher reaction temperature. There was no statistically significant difference in the DNG relative amount for the different process conditions. However, the process conditions of Samples 5 and 12 (e.g., a nitric acid to glycerol ratio of 4:1, a temperature of 35° C., and a 60 second residence time) produced the highest relative amount of DNG. The amount of NG was most sensitive to the nitric acid to glycerol ratio, and increased significantly when the nitric acid to glycerol ratio was increased from 4:1 to 8:1. Temperature and residence time did not have a significant effect on the amount of NG produced. The applied back flow pressure showed no statistical effects on the relative percentages of the reaction products.

### Example 3

### Glycerol Nitration

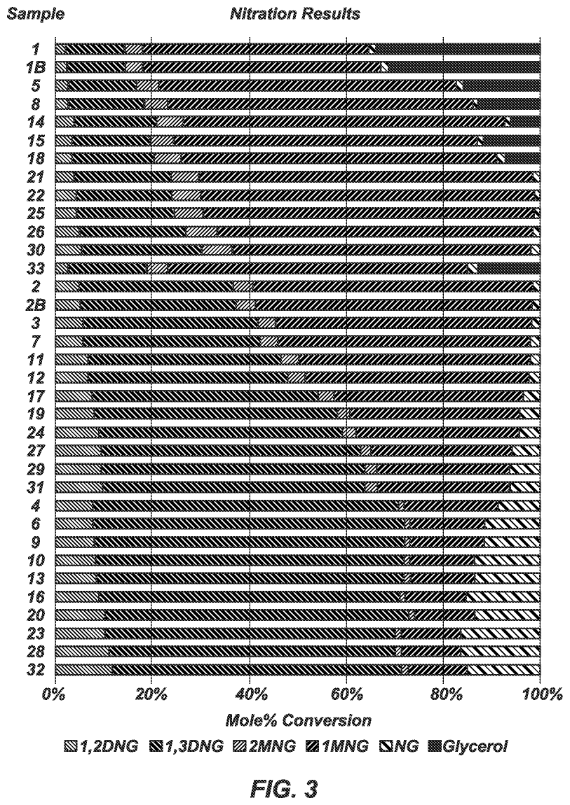

Glycerol and nitric acid were injected into the AFR described in Example 1. The glycerol and nitric acid were reacted at various process conditions and the reaction products in the product stream were analyzed to characterize the nitration of glycerol. Table 3 includes the process variables (reaction temperature (e.g., set temperature), ratio of nitric acid:glycerol, total input flow rate, and residence time) for each of Samples 1-33. FIG. 3 is a graphical representation of nitration results associated with Samples 1-33. The process variables for evaluating nitration of the glycerol were chosen based on the results in Example 2. The residence times included in Table 3 were calculated based on the AFR volume and total input flow rate. Actual residence times were approximately 5-10% longer than shown in Table 3.

TABLE 3
Sample Process Variables

| | | | Flow | Calculated |
|---|---|---|---|---|
| | Set Temp | HNO₃: | Rate | Residence |
| Sample | ° C. | Glycerol | (mL/min) | Time (Sec) |
| 1 | 35 | 2:1 | 2.000 | 60 |
| 1B | 35 | 2:1 | 2.000 | 60 |
| 5 | 35 | 2:1 | 1.333 | 90 |
| 8 | 35 | 2:1 | 1.000 | 120 |
| 14 | 45 | 2:1 | 1.333 | 90 |
| 15 | 45 | 2:1 | 2.000 | 60 |
| 18 | 45 | 2:1 | 1.333 | 90 |
| 21 | 45 | 2:1 | 1.000 | 120 |
| 22 | 55 | 2:1 | 2.000 | 60 |
| 25 | 55 | 2:1 | 2.000 | 60 |
| 26 | 55 | 2:1 | 1.333 | 90 |
| 30 | 55 | 2:1 | 1.000 | 120 |
| 33 | 35 | 2:1 | 2.000 | 60 |
| 2 | 35 | 3:1 | 2.000 | 60 |
| 2B | 35 | 3:1 | 2.000 | 60 |
| 3 | 35 | 3:1 | 1.333 | 90 |
| 7 | 35 | 3:1 | 1.333 | 90 |
| 11 | 35 | 3:1 | 1.000 | 120 |
| 12 | 45 | 3:1 | 2.000 | 60 |
| 17 | 45 | 3:1 | 1.333 | 90 |
| 19 | 45 | 3:1 | 1.000 | 120 |
| 24 | 55 | 3:1 | 2.000 | 60 |
| 27 | 55 | 3:1 | 1.333 | 90 |
| 29 | 55 | 3:1 | 1.000 | 120 |
| 31 | 55 | 3:1 | 1.000 | 120 |
| 4 | 35 | 4:1 | 2.000 | 60 |
| 6 | 35 | 4:1 | 1.333 | 90 |
| 9 | 35 | 4:1 | 1.333 | 90 |
| 10 | 35 | 4:1 | 1.000 | 120 |
| 13 | 45 | 4:1 | 2.000 | 60 |
| 16 | 45 | 4:1 | 1.333 | 90 |
| 20 | 45 | 4:1 | 1.000 | 120 |
| 23 | 55 | 4:1 | 2.000 | 60 |
| 28 | 55 | 4:1 | 1.333 | 90 |
| 32 | 55 | 4:1 | 1.000 | 120 |

As shown in FIG. 3, the concentration of reaction products in the product stream was most strongly affected by the ratio of nitric acid to glycerol. At a nitric acid to glycerol ratio of 4:1, the concentration of DNG compared to the other reaction products was high and remained similar for all examined combinations of other process conditions at the same nitric acid to glycerol ratio. At a nitric acid to glycerol ratio of 2:1, the concentration of DNG compared to the other reaction products was low and remained similar for other tested combinations of process conditions at the same nitric acid to glycerol ratio. However, at a nitric acid to glycerol ratio of 3:1, the concentration of DNG was maximized at higher temperatures and high nitric acid to glycerol ratios.

Glycerol was only detected in the product stream when the lowest nitric acid to glycerol ratio of 2:1 was used and at the low and mid temperatures of 35° C. and 45° C.

The concentration of the two isomers of MNG, 1-MNG and 2-MNG, were analyzed independently. The 1-MNG isomer was the major isomer in the product stream and was present at a concentration about 13 times higher than a concentration of the 2-MNG isomer. The concentration of MNG correlated strongly with the nitric acid to glycerol ratio, where a lower ratio of nitric acid to glycerol resulted in a higher concentration of MNG and a higher ratio of nitric acid to glycerol resulted in a lower concentration of MNG. Samples with ratios of nitric acid to glycerol of 2:1 and 3:1 showed some spread in the resulting concentrations of MNG based on reaction temperature. However, at the highest nitric acid to glycerol ratio of 4:1, there was little to no effect on the concentration of MNG based on the reaction temperature. According to these results, the most effective method of decreasing the concentration of MNG was to maximize the nitric acid to glycerol ratio at any of the tested temperatures. However, when a nitric acid to glycerol ratio of 3:1 was used, temperature became a more critical parameter and high temperatures could be used to minimize the concentration of MNG in the product stream.

The concentration of NG present in the product stream was strongly affected by the ratio of nitric acid to glycerol. A higher ratio of nitric acid to glycerol resulted in a higher concentration of NG in the product stream. The concentration of NG in the product stream was also affected by temperature, where high temperatures resulted in increased concentrations of NG.

The concentration of the two isomers of DNG, 1,2-DNG and 1,3-DNG, present in the product stream were analyzed independently. The 1,3-DNG isomer was the major isomer in the product stream and was present at a concentration about 6 times higher than a concentration of the 1,2-DNG isomer. The concentration of DNG was significantly affected by the nitric acid to glycerol ratio, where a higher ratio of nitric acid to glycerol resulted in higher concentrations of DNG in the product stream. The 1,2-DNG isomer showed a significant dependence on the reaction temperature. Higher reaction temperatures increased the concentration of the 1,2-DNG isomer in the product stream at all ratios of nitric acid to glycerol examined. The concentration of 1,3-DNG was independent from reaction temperature at the highest nitric acid to glycerol ratio of 4:1. Accordingly, when a nitric acid to glycerol ratio of 4:1 was used, temperature could be adjusted to accommodate other temperature dependent variables without affecting the concentration of 1,3-DNG in the product stream. It is possible to achieve conversions of glycerol to DNG above 70% by mole with the AFR configured as described in Example 1 and with certain process conditions (e.g., the process conditions used for analyzing Samples 4, 6, 9, 10, 13, 16, 20, 23, 28, and 32 shown in FIG. 3).

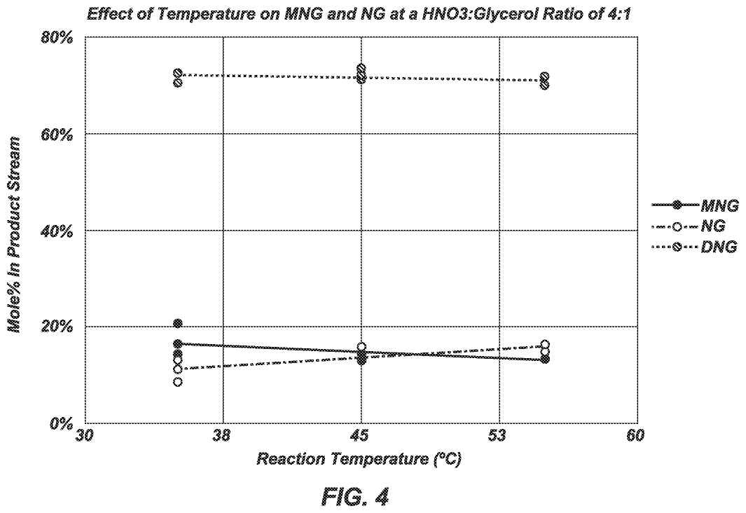

The independence of the 1,3-DNG concentration from reaction temperature at the highest nitric acid to glycerol ratio of 4:1 was surprising and unexpected. The independence of the 1,3-DNG concentration from reaction temperature at the highest nitric acid to glycerol ratio of 4:1 allowed for the concentrations of NG and MNG to be minimized by manipulating the reaction temperature without affecting the 1,3-DNG concentration. FIG. 4 depicts a plot of MNG and NG concentration plotted against reaction temperature at a constant nitric acid to glycerol ratio of 4:1. As shown in FIG. 4, concentrations of NG and MNG exhibited opposite trends with temperature and a cross-over occurring between 45° C. and 55° C. MNG is more easily removed from the product stream than NG. Therefore, optimal conditions may be those that minimize the concentration of NG in the product stream at the expense of increasing the concentration of MNG in the product stream.

### Example 4

### Glycerol Dilution

To test feasibility of using neat glycerol diluted with water to synthesize DNG, nitration was performed in batch using fuming nitric acid (90%) and neat glycerol. The nitration results of using neat glycerol were compared with nitration results of nitration performed in batch with glycerol diluted with 10 wt % and 15 wt % deionized water (DIW) (90% and 85% glycerol, respectively). It was surprisingly discovered that using the diluted glycerol did not decrease the yield of DNG produced in the nitration reaction. Rather, using 85% glycerol produced the highest levels of DNG while minimizing the amount of the unwanted side product MNG. In addition, 85% glycerol has a viscosity of 109 cP at 20° C., while neat glycerol has a viscosity of 1,400 cP at 20° C. The significantly reduced viscosity of the 85% glycerol enabled the diluted glycerol to be used in the microfluidic reactor.

Test nitrations were performed using an ADVANCED-FLOW® Reactor (AFR) from Dow Corning with microfluidic mixing channels (approximately 800 μm) and coolant-jacketed flow modules (e.g., plates) purchased from Dow Corning. Continuous peristaltic high-performance liquid chromatography (HPLC) pumps were used to pump reagents into the AFR and a chiller was used to pump coolant through the AFR at 400 mL/min to maintain a constant temperature. Nitration using 75% and 85% glycerol were tested in the AFR, and using both 75% and 85% glycerol achieved similar levels of nitration compared to those observed using neat glycerol. In the AFR, a 4:1 nitric acid to glycerol flow rate was used, with a residence time of only 60 seconds at 35° C. At these conditions, full conversion of the glycerol was achieved using both 75% and 85% glycerol, resulting in over 70% DNG yield and less than 15% conversion to NG. In contrast, when neat glycerol is used, 80% conversion of the glycerol occurred after 250 seconds of residence time, with 40% converted to NG.

### Example 5

### Microfluidic Reactor Configuration

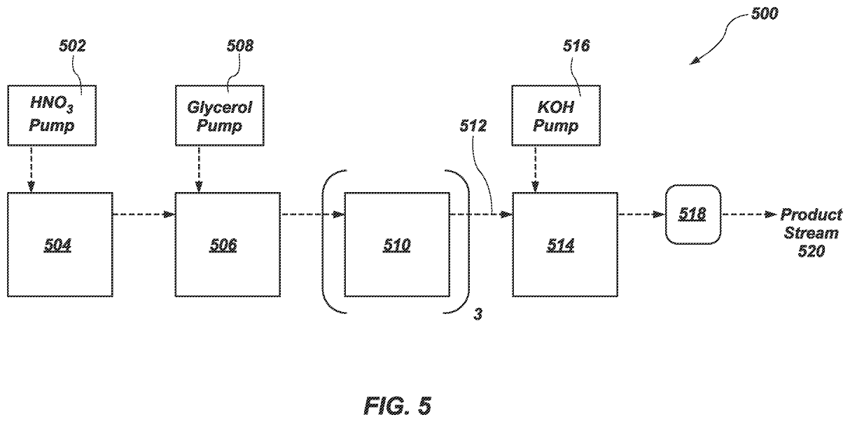

Referring to FIG. 5, a configuration 500 of an ADVANCED-FLOW® Reactor (AFR) from Dow Corning is shown. The AFR was used to synthesize GLYN by performing two reactions including a nitration reaction and an intramolecular ring closure reaction. As shown in FIG. 5, the AFR included a nitric acid pump 502 configured to pump nitric acid through a series of plates 504, 506, 510, 514. Nitric acid supplied by the nitric acid pump 502 entered into a temperature equilibration plate 504. Temperature equilibrated nitric acid from the temperature equilibration plate 504 then entered into a first mixing plate 506. A glycerol pump 508 was used to supply glycerol to the first mixing plate 506. The nitric acid and the glycerol were mixed in the first mixing plate 506 and then flowed through three residence plates 510 to form an intermediate product stream 512. The intermediate product stream 512 included reaction products of the nitration reaction. The intermediate product stream 512 was directed to a second mixing plate 514. A potassium hydroxide pump 516 was used to supply potassium hydroxide to the second mixing plate 514. The AFR was configured with a back pressure regulator 518, such that a product stream 520 from the second mixing plate 514 flowed through the back pressure regulator 518 after exiting the second mixing plate 514.

### Example 6

### GLYN Synthesis

Glycerol, nitric acid, and potassium hydroxide were injected into the AFR described in Example 5 to perform the two reactions, including the nitration reaction and the intramolecular ring closure reaction, to produce GLYN. A nitric acid to glycerol ratio of 4:1 and a residence time of greater than 60 seconds were used in the nitration reaction. A reaction temperature of 55° C. was determined based on the solubility of inorganic salts produced during acid neutralization. To accommodate flow of both the potassium hydroxide and the nitration reaction products, the nitration reaction products flow was set at 1 mL/min. Samples were collected for flow rates of 5 mL/min and 6 mL/min of 25 wt % potassium hydroxide at a reaction temperature of 55° C. The presence of GLYN in both samples was verified by NMR analysis. It was observed that using a lower reaction temperature, such as 35° C., resulted in a clog in the AFR, which was resolved by increasing the temperature back to 50° C.

### Example 7

### Microfluidic Reactor Configuration

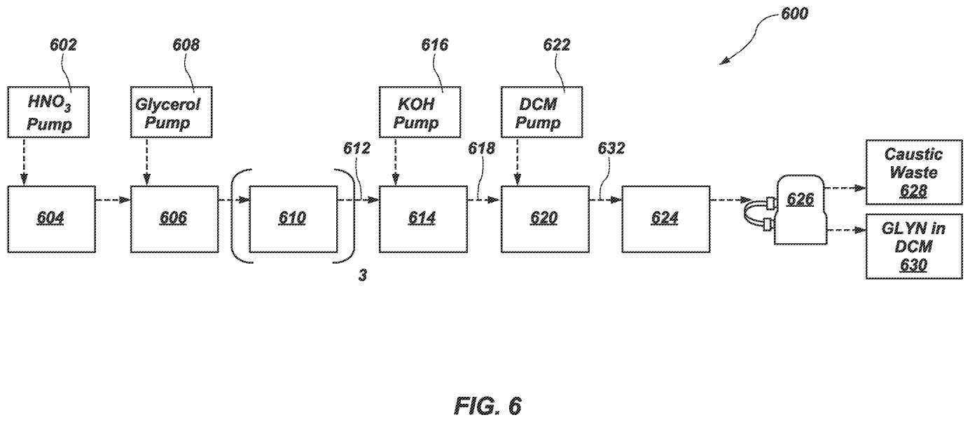

Referring to FIG. 6, a configuration 600 of an ADVANCED-FLOW® Reactor (AFR) from Dow Corning is shown. The AFR was used to synthesize GLYN by performing a two reactions including a nitration reaction and an intramolecular ring closure reaction. As shown in FIG. 6, the AFR included a nitric acid pump 602 configured to pump nitric acid through a series of plates 604, 606, 610, 614, 620, 624. Nitric acid supplied by the nitric acid pump 602 entered into a temperature equilibration plate 604. Temperature equilibrated nitric acid from the temperature equilibration plate 604 then entered into a first mixing plate 606. A glycerol pump 608 was used to supply glycerol into the first mixing plate 606. The nitric acid and the glycerol were mixed in the first mixing plate 606 and then flowed through three residence plates 610 to form an intermediate product stream 612. The intermediate product stream 612 included reaction products of the nitration reaction. The intermediate product stream 612 was directed to a second mixing plate 614. A potassium hydroxide pump 616 was used to supply potassium hydroxide to the second mixing plate 614 to perform the intramolecular ring closure reaction and form GLYN. A product stream 618 exited the second mixing plate 614 and entered a third mixing plate 620. A DCM pump 622 was used to supply DCM to the third mixing plate 620 to form a biphasic solution 632, including an organic phase and an aqueous phase. GLYN was extracted into the organic phase of the biphasic solution 632 and the nitric acid and other water-soluble byproducts were extracted into the aqueous phase of the biphasic solution 632. The biphasic solution 632 flowed through an additional residence plate 624 and into a phase-separator 626. The phase-separator 626 separated the organic phase and the aqueous phase of the biphasic solution 632, producing a caustic waste stream 628 and a product stream 630 including GLYN in DCM.

### Example 8

### GLYN Extraction

Glycerol, nitric acid, potassium hydroxide, and DCM were injected into the AFR described in Example 7 to perform two reactions, including a nitration reaction and an intramolecular ring closure reaction, and extract GLYN. Potassium hydroxide was tested as an alternative to sodium hydroxide. A nitric acid to glycerol ratio of 4:1 and a residence time of greater than 60 seconds were used in the nitration reaction. The reactions and extraction were performed at 55° C. The effects of potassium hydroxide concentration, caustic flow rate (e.g., potassium hydroxide flow rate), and DCM flow rate on the extraction of GLYN were evaluated. Table 4 includes the process variables (potassium hydroxide concentration, caustic flow rate, and DCM flow rate) and product conversion for each of Samples 1-5.

TABLE 4
Sample Process Variables and Results

| | Overall | | | | | MNG | GLYN | | |
|---|---|---|---|---|---|---|---|---|---|
| | How | Nitration | DCM Flow | KOH Flow | | Mole % | Mole % | DNG Mole | NG Mole |
| | Rate | Flow Rate | Rate | Rate | | Reaction | Reaction | % Reaction | % Reaction |
| Sample | (mL/min) | (mL/min) | (mL/min) | (mL/min) | [KOH] | Product | Product | Product | Product |
| 1 | 8 | 1 | 1 | 6 | 15% | 12% | 0% | 67% | 21% |
| 2 | 9 | 1 | 2 | 6 | 25% | 1% | 50% | 1% | 48% |
| 3 | 8 | 1 | 2 | 5 | 25% | 1% | 49% | 1% | 49% |
| 4 | 8 | 1 | 1 | 6 | 25% | 1% | 49% | 0% | 50% |
| 5 | 7 | 1 | 1 | 5 | 25% | 1% | 49% | 1% | 48% |

Samples 2-5 using 25% potassium hydroxide each produced similar results, with near complete conversion of DNG to GLYN and only trace amounts of MNG in the AFR. However, amounts of NG present in the product stream were high, likely due to the high nitration residence time and temperatures used. GLYN was successfully extracted into DCM, with an extraction residence time as little as 6.7 sec and a DCM:nitration products ratio as low as 1:1. However, the overall output of GLYN was relatively low (3.8 g/hr with the best process variables), with the reactor flow near its maximum.

### Example 9

### Microfluidic Reactor Configuration

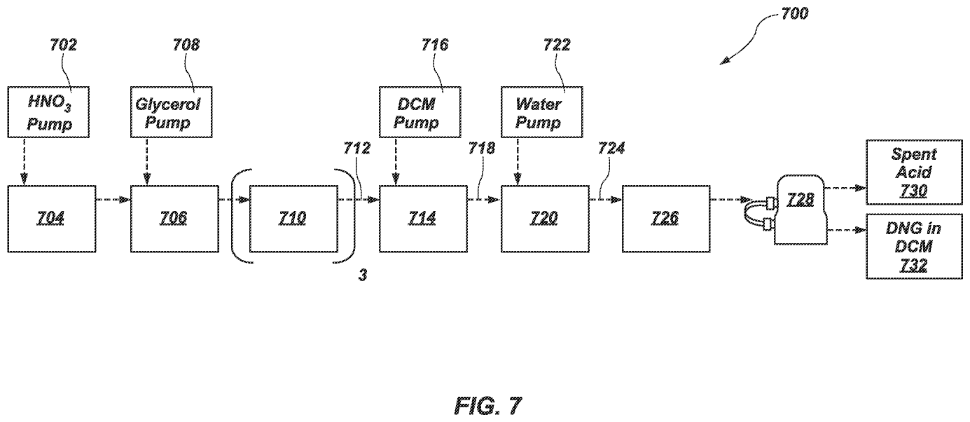

Referring to FIG. 7, a configuration 700 of an ADVANCED-FLOW® Reactor (AFR) from Dow Corning is shown. The AFR was used to perform a reaction process including a nitration reaction and separation of nitric acid from products of the nitration reaction. As shown in FIG. 7, the AFR included a nitric acid pump 702 configured to pump nitric acid through a series of plates 704, 706, 710, 714, 720, 726. Nitric acid supplied by the nitric acid pump 702 entered into a temperature equilibration plate 704. Temperature equilibrated nitric acid from the temperature equilibration plate 604 then entered into a first mixing plate 706. A glycerol pump 708 was used to supply glycerol into the first mixing plate 706. The nitric acid and the glycerol were mixed in the first mixing plate 706 and then flowed through three residence plates 710 to form an intermediate product stream 712. The intermediate product stream 712 included reaction products of the nitration reaction. The intermediate product stream 712 was directed to a second mixing plate 714. A DCM pump 716 was used to supply DCM to the second mixing plate 714 to form an intermediate solution 718, including the nitration products dissolved in the DCM. The intermediate solution 718 was directed to a third mixing plate 720. A pump 722 was used to supply water or a solution of an inert salt to the third mixing plate 720 to form a biphasic solution 724, including an organic phase and an aqueous phase. DNG was extracted into the organic phase of the biphasic solution 724 and the nitric acid and other water-soluble byproducts were extracted into the aqueous phase of the biphasic solution. The biphasic solution 724 flowed through an additional residence plate 726 and into a phase-separator 728. The phase-separator 728 separated the organic phase and the aqueous phase of the biphasic solution 724, producing a spent acid stream 730 and a product stream 732 including DNG in DCM.

### Example 10

### Pre-Separation of DNG from Nitric Acid

Glycerol, nitric acid, DCM, and water were injected into the AFR described in Example 9 to perform a nitration reaction and separate nitric acid from products of the nitration reaction. A nitration product flow rate was set to 2 mL/min and a DCM flow rate was set to 1 mL/min. At these flows, it was found that using a minimum of 1 mL/min of water induced flow to the non-wetting outlet of the separator. To aid diffusion of DNG into the DCM phase, replacing water with a solution of an inert salt (either Na₂SO₄ or NaCl) was also tested. The effects of water/salt solution flow rate, salt concentration, and DCM flow rate were evaluated. Table 5 includes the process variables (water/salt solution flow rate, salt concentration, and DCM flow rate) and DNG distribution in the product stream.

TABLE 5
Sample Process Variables and Results

| | DCM | | | DMG | DMG |
|---|---|---|---|---|---|
| | Flow | AQ: | Water/Salt | Fraction | Fraction |
| | Rate | DCM | Solution | in Non- | in |
| Sample | (mL/min) | Ratio | Added | Wetting | Wetting |
| 1 | 1 | 3:1 | DIW | 0.59 | 0.41 |
| 2 | 1 | 3:1 | 10% Na₂SO₄ | 0.55 | 0.45 |
| 3 | 2 | 3:2 | DIW | 0.30 | 0.70 |
| 4 | 2 | 3:2 | 15% Na₂SO₄ | 0.27 | 0.73 |
| 5 | 4 | 3:4 | DIW | 0.29 | 0.71 |
| 6 | 4 | 3:4 | DIW | 0.33 | 0.67 |
| 7 | 4 | 3:4 | 10% Na₂SO₄ | 0.31 | 0.69 |
| 8 | 4 | 3:4 | 15% Na₂SO₄ | 0.27 | 0.73 |

Nearly all of the MNG formed in the nitration reaction was found in the non-wetting (e.g., aqueous) phase of the biphasic solution and nearly all NG formed in the nitration reaction was found in the wetting (e.g., DCM, organic) phase of the biphasic solution. Glycerol was below the detection limit for all of the Samples. The DNG, however, was distributed between both the wetting and non-wetting phases. Under the conditions used to evaluate Samples 1-8, approximately 40%-70% of the DNG was extracted into the wetting phase. For aqueous phase to DCM (AQ:DCM) ratios less than or equal to 3:2, the extraction rate was very consistent, suggesting production of a reliable product stream even with small process fluctuations. Conducting a second separation could theoretically extract up to 90% of the DNG produced in the AFR.

Table 6 includes the total distribution of reaction products in the AFR for each of Samples 1-8 previously described in Table 5.

TABLE 6
Total Distribution of Reaction Products

| | Nitration Reaction | | | Wetting Phase | |
|---|---|---|---|---|---|
| | Products in AFR | | | Overall Output | |
| | DNG | | | DNG | NG |
| | Mole | MNG | NG | Output | Output |
| Sample | % | Mole % | Mole % | (g/hr) | (g/hr) |
| 1 | 54% | 4% | 42% | 9.2 | 15.9 |
| 2 | 73% | 14% | 13% | 11.2 | 3.9 |
| 3 | 51% | 2% | 46% | 26.4 | 33.6 |
| 4 | 56% | 3% | 42% | 26.3 | 26.9 |
| 5 | 74% | 11% | 15% | 23.5 | 6.6 |
| 6 | 57% | 3% | 39% | 19.0 | 19.3 |
| 7 | 74% | 12% | 14% | 20.2 | 5.4 |
| 8 | 59% | 4% | 37% | 20.0 | 17.0 |

Conversion rates of glycerol to DNG are similar to those seen in Example 3. The mole percentages of MNG and NG produced are slightly lower and higher, respectively, than those seen in Example 3, likely due to the increased residence time. The addition of up to 15% sodium sulfate (Na₂SO₄) in the extraction solution did not appear to affect the distribution of nitration products or the overall output of DNG in the wetting phase.

With the process variables of Sample 5, about 23.5 g/hr of DNG was produced at a total AFR flow rate of 7 mL/min. The AFR has an additional 3 mL/min capacity, which allows for flexibility in the process parameters, allowing scaling of the nitration flow or increasing the flow rate of DCM to boost the total DNG output. Assuming full conversion of DNG to GLYN in the caustic act as demonstrated in Example 7, GLYN could be produced in 2-3 days of continuous operation of the AFR.

The non-wetting (e.g., aqueous) sample outputs were titrated for acid concentration and were estimated to contain between 8.3 M-12 M nitric acid. By approximating the total acid available in the system from the flow rate of fuming nitric acid, it was approximated that greater than 97% of the nitric acid was extracted to the non-wetting phase in all samples.

### Example 11

### Microfluidic Reactor Configuration

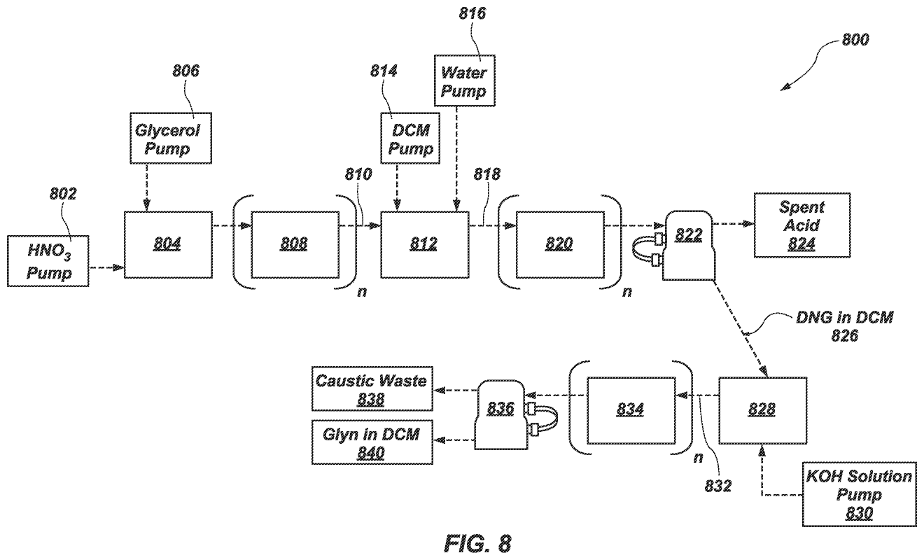

Referring to FIG. 8, a configuration 800 of an ADVANCED-FLOW® Reactor (AFR) from Dow Corning is shown. The AFR is used to synthesize GLYN by performing a reaction process including a nitration reaction, pre-separation of nitric acid from products of the nitration reaction, an intramolecular ring closure reaction, and separation of GLYN from byproducts of the reaction process. As shown in FIG. 8, the AFR is configured with a nitric acid pump 802 configured to pump nitric acid through the AFR. Nitric acid is supplied by the nitric acid pump 802 into a first mixing plate 804. A glycerol pump 806 is provided to supply glycerol into the first mixing plate 804. The nitric acid and the glycerol are mixed in the first mixing plate 804 and then flow through one or more residence plates 808 to form an intermediate product stream 810. The intermediate product stream 810 includes reaction products of the nitration reaction. The intermediate product stream 810 is directed to a second mixing plate 812. A DCM pump 814 and a water pump 816 are used to supply DCM and water, respectively, to the second mixing plate 812 to form an intermediate biphasic solution 818, including an aqueous phase and an organic phase. DNG from the reaction products of the nitration reaction is extracted into the organic phase of the intermediate biphasic solution 818. The intermediate biphasic solution 818 flows through one or more additional residence plates 820 and into a first phase-separator 822.

The first phase-separator 822 separates the aqueous phase and the organic phase of the intermediate biphasic solution 818 into a spent acid stream 824 and a product stream 826 including the DNG in DCM. The product stream 826 is directed to flow into a third mixing plate 828. A potassium hydroxide pump 830 is used to supply potassium hydroxide to the third mixing plate 828 to perform the intramolecular ring closure reaction and form a second intermediate biphasic solution 832 including an aqueous phase and an organic phase. The second intermediate biphasic solution 832 is directed through one or more additional residence plates 834 and into a second phase-separator 836. The second phase-separator 836 separates the aqueous and organic phases of the intermediate biphasic solution 832 into a caustic waste stream 838 and a product stream 840 including the GLYN in DCM.

While the disclosure is susceptible to various modifications and alternative forms, specific embodiments have been shown by way of example in the drawings and have been described in detail herein. However, the disclosure is not intended to be limited to the particular forms disclosed. Rather, the disclosure encompasses all modifications, equivalents, and alternatives falling within the scope of the disclosure as defined by the following appended claims and their legal equivalents.

What is claimed is:

1. A method of producing glycidyl nitrate, comprising:
reacting a glycerol solution and nitric acid in a microfluidic reactor to form a dinitroglycerol solution, the glycerol solution exhibiting a viscosity of less than or equal to about 150 cP at about 20° C.; and
reacting the dinitroglycerol solution with a base in the microfluidic reactor to form glycidyl nitrate.

2. The method of claim 1, wherein reacting a glycerol solution and nitric acid in a microfluidic reactor comprises reacting an aqueous glycerol solution and the nitric acid in the microfluidic reactor.

3. The method of claim 2, wherein reacting an aqueous glycerol solution and nitric acid in the microfluidic reactor comprises reacting the aqueous glycerol solution comprising from about 30% by weight glycerol to about 90% by weight glycerol and the nitric acid.

4. The method of claim 1, further comprising removing excess nitric acid from the dinitroglycerol solution before reacting the dinitroglycerol with the base.

5. The method of claim 4, wherein removing excess nitric acid from the dinitroglycerol solution comprises extracting the excess nitric acid from the dinitroglycerol solution.

6. The method of claim 1, further comprising recovering the dinitroglycerol into an organic solvent before reacting the dinitroglycerol with the base.

7. The method of claim 6 wherein recovering the dinitroglycerol into an organic solvent comprises extracting the dinitroglycerol into dichloromethane before reacting the dinitroglycerol with the base.

8. The method of claim 1, wherein reacting the dinitroglycerol solution with a base in the microfluidic reactor to form glycidyl nitrate comprises reacting the dinitroglycerol solution with potassium hydroxide.

9. The method of claim 1, wherein reacting the glycerol solution and nitric acid in a microfluidic reactor comprises reacting the glycerol solution and a nitric acid solution comprising the nitric acid and a solvent comprising water or dichloromethane.

10. The method of claim 9, wherein reacting the glycerol solution and a nitric acid solution comprises reacting the glycerol solution and the nitric acid solution comprising about 90 wt % nitric acid and about 10 wt % water.

11. A method of producing glycidyl nitrate, comprising:
reacting an aqueous glycerol solution and nitric acid in a microfluidic reactor at a temperature between about 15° C. and about 55° C. to form a dinitroglycerol solution; and
reacting the dinitroglycerol solution with an aqueous potassium hydroxide solution in the microfluidic reactor at a temperature between about 20° C. and about 60° C. to form glycidyl nitrate.

12. The method of claim 11, wherein reacting the dinitroglycerol solution with an aqueous potassium hydroxide solution comprises reacting the dinitroglycerol solution with the aqueous potassium hydroxide solution comprising potassium hydroxide at a concentration of about 7.2 M.

13. The method of claim 11, wherein reacting an aqueous glycerol solution and nitric acid in a microfluidic reactor comprises reacting the aqueous glycerol solution exhibiting a viscosity of less than or equal to about 150 cP at 20° C. and the nitric acid.

14. The method of claim 11, wherein:
reacting an aqueous glycerol solution and nitric acid in a microfluidic reactor comprises reacting the aqueous glycerol solution and the nitric acid in the microfluidic reactor at a temperature between about 20° C. and about 35° C.; and
reacting the dinitroglycerol solution with an aqueous potassium hydroxide solution in the microfluidic reactor comprises reacting the dinitroglycerol solution with the aqueous potassium hydroxide solution in the microfluidic reactor at a temperature between about 50° C. and about 60° C.

15. A system for producing glycidyl nitrate, comprising:
a microfluidic reactor comprising one or more inlets configured to introduce diluted glycerol, nitric acid, potassium hydroxide, and dichloromethane into channels thereof, the diluted glycerol exhibiting a viscosity of less than or equal to about 150 cP at about 20° C.;
one or more liquid:liquid phase separators coupled to the microfluidic reactor and configured to remove nitric acid from a biphasic dinitroglycerol solution comprising dinitroglycerol, nitric acid, dichloromethane, and water; and
one or more additional liquid:liquid phase separators coupled to the microfluidic reactor and configured to recover glycidyl nitrate from a biphasic glycidyl nitrate solution comprising glycidyl nitrate and dichloromethane.

16. The system of claim 15, further comprising one or more pumps configured to separately introduce the diluted glycerol and the nitric acid.

17. The system of claim 15, further comprising one or more pumps configured to separately introduce the potassium hydroxide and the dichloromethane.

18. The system of claim 15, wherein a first channel of the microfluidic reactor is configured to react the diluted glycerol and the nitric acid.

19. The system of claim 15, wherein a second channel of the microfluidic reactor is configured to react the dinitroglycerol and potassium hydroxide.

20. The system of claim 15, wherein a reaction volume of the microfluidic reactor comprises less than about 40 ml and an inner diameter of a reaction channel of the microfluidic reactor is less than or equal to about 1000 μm.
